(19) United States
(12) Patent Application Publication
(10) Pub. No.: US 2018/0071012 A1
(43) Pub. Date: Mar. 15, 2018

(54) METHOD OF PERFORMING ARTHROSCOPIC SURGERY

(71) Applicant: Cannuflow, Inc., Campbell, CA (US)

(72) Inventor: Theodore R. Kucklick, Campbell, CA (US)

(73) Assignee: Cannuflow, Inc., Campbell, CA (US)

(21) Appl. No.: 15/706,275

(22) Filed: Sep. 15, 2017

Related U.S. Application Data
(60) Provisional application No. 62/395,264, filed on Sep. 15, 2016.

(51) Int. Cl. A61B 18/14; A61B 17/3207; A61B 17/32; A61B 1/317

(52) U.S. Cl. CPC A61B 18/148; A61B 17/3207; A61B 17/0218; A61B 1/317; A61B 17/320068

(57) ABSTRACT

Systems and method for cooling tissue in an arthroscopic radio frequency ablation surgical procedure in order to prevent tissue damage.

This application claims priority to U.S. Provisional Application 62/395,264 filed Sep. 15, 2016.

### FIELD OF THE INVENTIONS

The inventions described below relate to the field of arthroscopic surgical procedures and more specifically, to radio frequency ablation procedures.

### BACKGROUND OF THE INVENTIONS

Radio frequency ablation surgery performed in low fluid volume spaces can have catastrophic complications. For a shoulder surgery with a start temperature of 25° C., a shoulder Sub Acromial (SA) space of 25 ml, and a nominal application of RF power, the temperature after three minutes is 42° C. For a hip surgery with a start temperature of 25° C., a hip central compartment of 6.5 ml, and a nominal application of RF power, the temperature after three minutes is 65° C. Fluid temperatures above 45° C. kill cartilage. Studies have shown that third-degree burns can occur in as little as two seconds at 65° C., fifteen seconds at 56.1° C., and five minutes at 50° C. All of these are attainable temperatures in the course of hip arthroscopy. Conventional bipolar wand technology cannot safely control temperature in hip joints. This tissue must be actively cooled.

Another complication associated with RF surgery is the risk of hypothermia to the patient caused by injection of the cold fluid needed to prevent overheating of the joint. Operating rooms are often kept very cold (15-20° C.). In surgery, a high volume of “room temperature” (15-20° C.) fluid is injected into a joint space in order to prevent the buildup of RF heat in the surrounding tissue. If the patient's core temperature drops below 35° C. due to the “room temperature” fluid there is a high risk of hypothermia. To reduce the risk of hypothermia, surgeons warm the cooling fluid, however this correspondingly increases the risk of thermal damage to tissue surrounding the joint space.

The high volume of cooling fluid used in these procedures is also very wasteful and expensive. In current surgical procedures, four or five 3-liter bags of fluid are used, with each bag costing about $25. This is a direct cost of up to $125 just in fluid per procedure.

### SUMMARY

The systems and methods described below provide for cooling tissue in an arthroscopic radio frequency ablation surgical procedure. The system includes a fluid chilling assembly that maintains fluid at a cooled temperature and delivers the cooled fluid to the surgical site while the RF ablation tool is energized and suspends cooling flow when the tool is not energized. The fluid bag holder has a Peltier module or other cooling means to maintain the fluid bag at the chilled temperature. A fluid pump pumps fluid from the chilled fluid bag through a fluid outflow tube. A sensor and a control system are provided to turn the pump on to initiate flow when the RF is energized and turn the pump off to stop flow when the RF tool is de-energized.

The sensor used to actuate the pump can be with an induction connection, which senses RF in the cable to the RF wand. This eliminates the need to have special connectors and adapters, and allows the unit to connect easily to any existing RF system.

Cooled fluid is delivered when the RF is active and shuts off when it is not. The system only needs to supply 100 mL or less to the joint space, and only when RF is applied. The subsequent reduction in the volume of cold fluid minimizes the risk of hypothermia. It also reduces the risk to the patient from extravasation.

The chilled fluid can run through a dedicated channel in the RF wand, through the existing fluid channel in the RF wand, through an inflow/outflow sheath or through a dedicated chilled fluid inflow cannula.

### BRIEF DESCRIPTION OF THE DRAWINGS

FIGS. 1a and 1b illustrate the insulated fluid chilling assembly.

FIG. 2 illustrates the system.

### DETAILED DESCRIPTION OF THE INVENTIONS

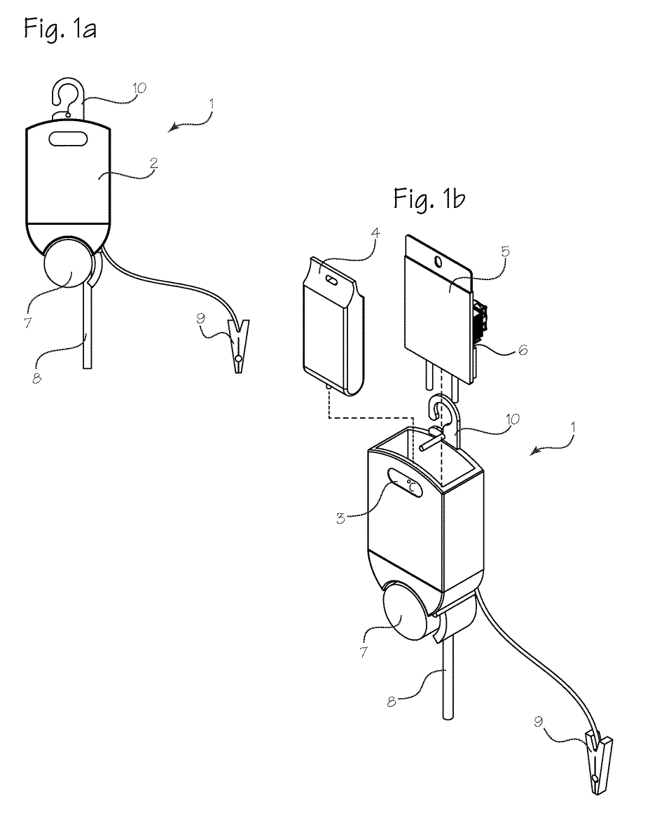

FIGS. 1a and 1b illustrate the insulated fluid chilling assembly 1. The insulated fluid bag holder 2 is provided with a display 3 for reporting the fluid temperature, fluid pressure, flow rates, etc. A chilled fluid bag 4 is inserted into the fluid bag holder 2. A Peltier module 5, cooling plate 6 and insulation inside the fluid bag holder keep the fluid bag contents cool. A peristaltic fluid pump 7 pumps fluid from the fluid bag through a fluid outflow tube 8. A sensor 9 is connected to the RF ablation device or foot pedal (or both) for sensing when the RF tool is activated. A control system is provided to operate the pump to initiate flow when the sensor detects that the RF tool is energized. The sensor can be an induction sensor or may be electrically connected to the foot switch or other RF actuation. A hook 10 is provided for hanging the fluid bag holder 2 on an IV Pole.

Preferably the fluid bag 4 has been pre-chilled prior to the surgical procedure and thus, the fluid bag holder will act to maintain the temperature 5-10° C. for an extended time rather than act to chill the fluid to desired temperature of 5-10° C. The fluid bag holder should be designed to hold at least a 3-liter bag of fluid.

Other cooling devices besides Peltier module may be used, so long as the other cooling device is operable to maintain the bag fluid at the 5-10° C. temperature. For example, an ice pack, gel pack or other portable sac filled with water, refrigerant gel or liquid may be frozen and used in place of the Peltier plate. The fluid bag holder 2 walls may be hollowed and filled with water or gel and the entire fluid bag holder put in a freezer and when in use, the fluid bag can be dropped into the fluid bag holder 2 and connected to the pump at time of surgery.

The small integrated peristaltic pump 7 in the fluid bag holder 2 is operable to deliver fluid in the range of 1-500 mL per minute depending on the RF procedure being performed, and pump cold fluid into the joint space to prevent the fluid in the joint space from getting hot and thus keep heat from building up in the surrounding tissue. The flow rate is variable depending on the joint being operated upon and thus there can be several flow rate settings based on the size of the joint space as follows: flow rates of 5 mL to 50 mL/min for small joints (wrist/ankle/elbow); flow rates of 5 mL to 100 mL/min for hip joints; flow rates of 5 mL to 200 mL/min for shoulder joints; and flow rates of 5 ml to 300 mL/min for knee joints.

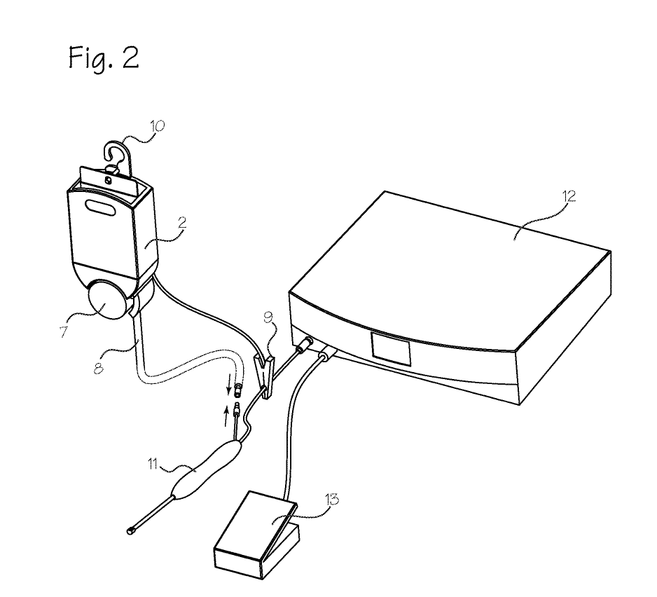

FIG. 2 shows the system, including the chilling assembly 1, sensor 9, an RF ablation device 11, RF generator 12, and a foot pedal 13. The system is actuated by pressing the pedal for the RF wand.

The sensor is attached to an external portion of the RF wand, such as the cable, the handle, or a proximal portion of the cable, and is preferably releasably attached to the RF wand or a component of the wand (i.e., it may be readily attached and detached by hand, without the use of tools) with a spring clip, as shown or with any other releasable attachment means such as a resiliently expandable sleeve, a snap-fit sleeve, etc. The releasable attachment means is preferably configured for releasable attachment to the cable extending from the RF power supply 12 to the RF wand 11. This allows the system to control cooling fluid flow, responsive and tied to the application of RF power to the joint, in conjunction with any existing RF power supply and RF ablation tool, without the need to integrate the cooling system with the RF ablation system. Though not so convenient, the sensor may be attached to the RF wand or a component such as the RF cable with screw-on clamps, to provide the advantage of a cooling system universally applicable to any RF ablation system. In either case, the attachment of the sensor to an external surface of a component of the RF ablation tool allows for use of the cooling system with any RF ablation system, without the need to integrate the two systems further.

The sensor operable to detect the passage of RF energy to the RF wand is most conveniently provided in the form of a current sensor, but any sensor operable to detect the passage of RF energy to the wand may be used. Current sensors such as Hall effect sensors, Rogowski coils, current transformers, Magnetoresistive sensors and Fluxgate sensors, or any other current sensor may be used. The sensor need only detect the presence or absence of RF power through the cable. EMF sensors that detect the electromagnetic field associated with the passage of RF energy, not strictly referred to as current sensors, may be used.

The conduit may feed into a lumen of a sheath used in conjunction with the RF wand, or it may communicate with a fluid lumen in the RF wand itself, or it may communicate with a fluid supply cannula separate from the sheath and wand, which is inserted into the arthroscopic workspace through a separate portal.

The control system is operable to receive input from the sensor, such as a signal indicative of the application of RF power, and control the pump to provide cooling fluid flow to the surgical site in response to the signal indicative of the application of RF power indicates that RF power is applied to the wand, and cease cooling fluid flow when the signal indicative of the application of RF power indicates that RF power is not applied to the wand.

The control system may comprise at least one processor and at least one memory including program code with the memory and computer program code configured with the processor to cause the system to perform the functions described throughout this specification. The various functions of the control system may be accomplished in a single computer or multiple computers, and may be accomplished by a general purpose computer or a dedicated computer, and may be housed in the housing or an associated housing.

The system described above is useful in orthopedic procedures as well as gynecology and urology procedures where protection from RF heat is needed. It is also useful in other heat producing procedures, such as ultrasonic scalpels, or procedures involving drilling or sawing bone. In addition to preventing RF heat damage, the cooled fluid technology helps constrict blood vessels, assists hemostasis, and cold fluid provides analgesic effect.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

I claim:

1. A cooling system for cooling a joint during arthroscopic surgery involving the application of RF power to body tissue within an arthroscopic work space using an RF ablation tool, said cooling system comprising:
a reservoir of cooling fluid suitable for injection to an arthroscopic workspace;
a conduit for conducting the cooling fluid to the arthroscopic workspace, said conduit in fluid communication with the reservoir;
a pump for pumping the fluid from the reservoir to the arthroscopic workspace through the conduit;
a sensor for sensing flow of RF power through an RF ablation tool, said sensor operable to generate and transmit a signal indicative of the flow of RF power through the RF ablation tool;
a control system for operating the pump to supply cooling fluid to the arthroscopic work space in response to sensed application of RF power to the RF ablation tool, wherein said control system is operable to receive a signal from the sensor, indicative of the application of RF power, and control the pump to provide cooling fluid flow to the surgical site when the signal indicative of the application of RF power indicates that RF power is applied to the RF ablation tool, and cease cooling fluid flow when the signal indicative of the application of RF power indicates that RF power is not applied to the RF ablation tool;
wherein the sensor is configured for attached to an external surface of a component of the RF ablation tool.

2. The cooling system of claim 1 wherein the sensor is configured for releasable attachment to an external surface of a component of the RF ablation tool.

3. The cooling system of claim 1 further comprising releasable attachment means configured for releasable attachment of the sensor to a cable of the RF ablation tool that conducts RF power from and RF power supply to an RF ablation tool.

4. The cooling system of claim 1 further comprising a cooling system for cooling the fluid in the reservoir.
